(19) United States
(12) Patent Application Publication
(10) Pub. No.: US 2023/0175029 A1
(43) Pub. Date: Jun. 8, 2023

(54) ENZYME CASCADES BASED ON SUCROSE SYNTHASE AND PYROPHOSPHORYLASE FOR CONVERSION OF ADP TO ATP

(71) Applicant: RHEINISCH-WESTFALISCHE TECH-NISCHE HOCHSCHULE (RWTH) AACHEN, Aachen (DE)

(72) Inventors: Prof. Dr. Lothar ELLING, Aachen (DE); Johannes GOTTSCHALK, Aachen (DE); Hannes FROHNMEYER, Aachen (DE)

(21) Appl. No.: 17/997,364

(22) PCT Filed: Apr. 21, 2021
(86) PCT No.: PCT/EP2021/060401

§ 371 Date: Oct. 27, 2022

(30) Foreign Application Priority Data
Apr. 28, 2020 (DE) 10 2020 111 560.1

(51) Int. Cl. C12P 19/32; C12P 19/02; C12N 9/12

(52) U.S. Cl. CPC C12P 19/32; C12P 19/02; C12N 9/1229

(57) ABSTRACT

The present invention relates to a process for the multi-step enzymatic conversion of adenosine diphosphate (ADP) to adenosine triphosphate (ATP), the process comprising the steps of: a) enzyme-catalyzed conversion of adenosine diphosphate in the presence of sucrose and a sucrose synthase to adenosine diphosphate-glucose; and b) enzyme-catalyzed conversion of the adenosine diphosphate-glucose formed in process step a) in the presence of inorganic pyrophosphate and a pyrophosphorylase to adenosine triphosphate and glucose-1-phosphate. Furthermore, the invention relates to the use of the process for the preparation of sugar phosphates, nucleotide sugars, glycans, glycoproteins, glycolipids or glycosaminoglycans.

The present invention relates to a process for the multi-step enzymatic conversion of adenosine diphosphate (ADP) to adenosine triphosphate (ATP), the process comprising the steps of: a) enzyme-catalyzed conversion of adenosine diphosphate in the presence of sucrose and a sucrose synthase to adenosine diphosphate-glucose; and b) enzyme-catalyzed conversion of the adenosine diphosphate-glucose formed in process step a) in the presence of inorganic pyrophosphate and a pyrophosphorylase to adenosine triphosphate and glucose-1-phosphate. Furthermore, the invention relates to the use of the process for the preparation of sugar phosphates, nucleotide sugars, glycans, glycoproteins, glycolipids or glycosaminoglycans.

The efficient and reproducible synthesis of defined macromolecules is still a major challenge for science and industry. Although possible solutions can be given in principle for a large number of syntheses, the successful transfer to the large scale, under the boundary conditions of a cost-effective and high-yield production, is not feasible in all cases. This applies in particular to biological macromolecules whose functionality is based not only on their actual composition but also on a specific, three-dimensional structure. A representative of this substance class is hyaluronic acid (hyaluronan, HA), a non-sulfated glycosaminoglycan (GAG) composed of repeating disaccharide units of β1,4-D-glucuronic acid and β1,3-N-acetyl-D-glucosamine (4GlcAβ1-3GlcNAcβ1-). The HA represents the main component of the extracellular matrix (ECM) and a single molecule can reach a molecular weight (MW) of up to 10 MDa. While a variety of biological manufacturing processes have been successfully brought to market in recent years, these processes still had the disadvantage that HA with only relatively “low” molecular weights, for example up to 500 kDa, can be provided in an economically reasonable way. In particular, higher molecular weights with a defined molecular weight distribution are currently not available in larger quantities at reasonable cost. One of the reasons for the latter is that expensive starting materials, such as adenosine triphosphate (ATP), have to be used for HA, which is usually obtained via fermentation.

In biotechnology, adenosine triphosphate (ATP) is widely used in the synthesis of fine chemicals, pharmaceuticals, or biopolymers by biotransformation and biocatalysis. Since ATP is a major cost factor, regeneration of spent ATP is sought. On the one hand, this can reduce costs and, on the other hand, alleviate the limitations of ATP-dependent reactions.

The most commonly used method for ATP regeneration involves the phosphorylation of ADP with phosphoenolpyruvate (PEP) by a pyruvate kinase (PK). However, this system is subject to limitations because pyruvate is released in this reaction, which has an inhibitory effect on PK. This release affects the overall efficiency of ATP regeneration. Second, PEP is a very expensive substrate and more expensive than ATP, which directly reduces the economics of ATP regeneration. Recent approaches to ATP regeneration utilize polyphosphates (PolyP) and polyphosphate kinases (PPK). Long-chain polyPs are used to regenerate ATP from ADP with high efficiency. However, this system is hampered by undefined chain lengths of polyPs, since polyphosphates cannot be obtained in uniform chain lengths so far. Therefore, the achievable ATP regeneration cycles vary. Furthermore, polyphosphates cannot be completely degraded by PPKs, resulting in contamination of the synthesis approach with polyphosphate residues. These in turn have to be degraded or separated from the product. The possibility of using PPᵢ directly for ATP synthesis has not yet been described for this enzyme class.

The patent literature also contains a wide variety of approaches to enzymatic conversions of ATP derivatives or degradation products such as ADP.

For example, DE 60 2005 026 917 D1 discloses a method for producing recombinant sucrose synthase, use thereof in the production of kits for sucrose determination, as well as methods for producing ADP-glucose and methods for obtaining transgenic plants with leaves and storage organs in which ADP-glucose and starch accumulate in high concentration.

Furthermore, CN 101 294 167 A discloses a method for producing hydrogen by culturing hydrogen-producing microbes in a culture medium containing phosphate oils. In this writing, phosphate is added in a cellular environment to simultaneously promote rapid growth and extraction of ATP, thereby controlling ATP levels and oxidation-reduction potential in cells and accelerating carbon source consumption and production efficiency and hydrogen. In addition, the phosphate can be recycled in the culture system without contaminating the environment.

In another patent document, U.S. Pat. No. 2,007,005 4283 A1, a low-cost DNA sequencing method with high sensitivity is provided. The method comprises the steps of adding a given amount of dATP for stepwise complementary strand synthesis and subtracting the background luminescence intensity caused by dATP from the measured luminescence intensity to obtain the luminescence intensity involved in complementary strand synthesis.

Such solutions known from the prior art may offer further potential for improvement, in particular with regard to the controlled enzymatic provision of ATP within complex conversions by the work-up of ATP degradation products, whereby the conversions can be used to a high degree of flexibility in larger enzyme cascades.

It is therefore the task of the present invention to at least partially overcome the disadvantages known from the prior art. In particular, it is the task of the present invention to provide an improved process and an improved use in which ATP is recovered enzymatically from ATP degradation products, whereby the work-up also takes place in complex reaction environments with further enzymes and reactants without significant interactions.

The task is solved by the features of the independent claims, directed to the method according to the invention and the use of the method according to the invention. Preferred embodiments of the invention are indicated in the dependent claims, in the description or in the figures, whereby further features described or shown in the dependent claims or in the description or in the figures may individually or in any combination constitute an object of the invention, as long as the context does not clearly indicate the contrary.

According to the invention, the problem is solved by a process for the multistage enzymatic conversion of adenosine diphosphate to adenosine triphosphate, the process comprising at least the steps:

(a) enzyme-catalyzed conversion of adenosine diphosphate in the presence of sucrose and a sucrose synthase to adenosine diphosphate-glucose; and
b) enzyme-catalyzed conversion of the adenosine diphosphate-glucose formed in process step
a) in the presence of inorganic pyrophosphate and a pyrophosphorylase to adenosine triphosphate and glucose-1-phosphate;
wherein process steps a) and b) are carried out in aqueous solution and simultaneously or successively.

Surprisingly, it was found that by means of a combination of a sucrose synthase (Sucrose Synthase, SuSy) and an ADP-glucose pyrophosphorylase (AGPase), ATP can be reformed from sucrose (Suc) and inorganic pyrophosphate (PPᵢ) from ADP. The enzyme cascade according to the invention does not generate free phosphate (Pᵢ) and the 2-step cascade is also robust and flexible to a high degree, so that it can be combined with other or further enzyme cascades in which ATP is required (e.g. kinases). In addition, these substeps can be used in combination with enzyme cascades in which PPᵢ is formed, which can then be removed from equilibrium and efficiently used for ATP synthesis. The reactants used are inexpensive compared to the reactants proposed in the prior art and the individual process steps can be run together or independently in wide temperature and concentration parameter ranges. Thus, compared to the prior art, the system is capable of performing ATP regeneration from PPᵢ and sucrose in multiple cycles in a pyrophosphate (PPᵢ) releasing process. The energy required for this is obtained by adding sucrose and PPᵢ, which on the one hand reduces the amount of total ATP to be added and on the other hand keeps the process phosphate-free. Furthermore, the system can also be used for processes that do not release PPᵢ by separately adding PPᵢ to them. In this case, the amount of regenerated ATP is equivalent to the amount of PPᵢ used. This can also significantly reduce the amount of ATP to be used in the reaction. The enzyme cascade can be easily combined with ATP-consuming syntheses and applied to ATP regeneration from the inexpensive and stable substances sucrose and PPᵢ. The enzyme cascade does not release phosphate, which favors large-scale synthesis and product purification. For example, no magnesium phosphate is produced. Due to the favorable market prices of the energy suppliers, this conversion is particularly suitable for large-scale in vitro syntheses, which can also be designed particularly simply due to the possibility of simple process control in only one reaction solution.

The process according to the invention is a process for the multi-step enzymatic conversion of adenosine diphosphate to adenosine triphosphate. The conversion of ADP to ATP thus takes place via the use of at least two, different enzymes, whereby the ATP is not formed within a single step, but via several steps, i.e. via at least one, potentially isolatable, intermediate product. Within the conversion, ADP according to the following structural formula

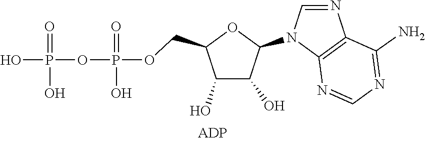 [SMILES: Nc1ncnc2c1ncn2[C@@H]1O[C@H](COP(=O)(O)OP(=O)(O)O)[C@@H](O)[C@H]1O]

in converted into ATP with the following structural formula

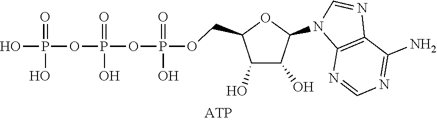 [SMILES: Nc1ncnc2c1ncn2[C@@H]1O[C@H](COP(=O)(O)OP(=O)(O)OP(=O)(O)O)[C@@H](O)[C@H]1O]

at the catalytic centers of at least two enzymes.

The process comprises the process step a) of enzyme-catalyzed conversion of adenosine diphosphate in the presence of sucrose and a sucrose synthase (SuSy) to adenosine diphosphate-glucose. The first step is carried out by a bacterial or plant sucrose synthase in an aqueous solution. For the reaction, the presence, addition or formation of sucrose in the solution is mandatory and the activity of the synthase converts the ADP to adenosine diphosphate-glucose (ADP-Glc) with degradation of sucrose (Suc) to fructose (Fru). The reaction in the first substep can be represented by the following partial reaction equation:

ADP+Suc↔ADP-Glc+Fru.

The reaction substep can be carried out at a temperature greater than or equal to 15° C. and less than or equal to 50° C., preferably greater than or equal to 25° C. and less than or equal to 40° C. The reaction solution may be pH buffered via a buffer system. SuSy concentrations may be greater than or equal to 0.001 μg/L and less than or equal to 5 μg/L, preferably from greater than or equal to 0.01 μg/L and less than or equal to 1 μg/L. Preferred concentration of Suc can be greater than or 50 mM and less than or equal to 500 mM, preferably greater than or 100 mM and less than or equal to 350 mM.

The process comprises process step b) of enzyme-catalyzed conversion of the adenosine diphosphate-glucose (ADP-Glc) formed in process step a) in the presence of inorganic pyrophosphate (PPᵢ) and a pyrophosphorylase (AGPase) to adenosine triphosphate (ATP) and glucose-1-phosphate (Glc-1-P). The second step is carried out by an AGPase in an aqueous solution. For the reaction, the presence, addition or formation of PPᵢ in the solution is mandatory and the activity of the AGPase converts the ADP-Glc to Glc-1-P and ATP consuming the PPᵢ. The reaction in the second substep can be represented by the following partial reaction equation:

ADP-Glc+PPᵢ↔Glc-1-P+ATP.

The reaction substep can be carried out at a temperature greater than or equal to 15° C. and less than or equal to 50° C., preferably greater than or equal to 25° C. and less than or equal to 40° C. The reaction solution may be pH buffered via a buffer system. AGPase concentrations may be greater than or equal to 500 μg/L and less than or equal to 5000 μg/L, preferably from greater than or equal to 1000 μg/L and less than or equal to 4000 μg/L. Preferred PPᵢ concentrations can be greater than or 0.1 mM and less than or equal to 100 mM, preferably greater than or 1 mM and less than or equal to 35 mM.

In sum, both enzymatic process steps can be described by the following reaction equation:

ADP+Suc+PPᵢ↔ATP+Fru+Glc-1-P.

Process steps a) and b) are carried out in aqueous solution and can be performed simultaneously or sequentially. The multistage reaction can take place in a single solution or in separate solutions, in which case partial amounts of the liquids are exchanged. The process control can be designed in such a way that the reactants are present in the reaction solution from the beginning of the reaction. However, it is also possible that the reactants and/or the enzymes are added during the course of the reaction. Furthermore, it is possible that the substances necessary for the synthesis performance of the enzymes are only formed by other reactions in the course of the reaction. For this purpose, these further reactions can take place at other reaction sites or in one and the same solution of process steps a) and/or b). The two enzymes can move “freely” in the reaction solution or one or both enzyme systems can be bound to or on carriers in the solution.

In a preferred embodiment of the process, process steps a) and b) can be carried out in a common aqueous reaction solution. For an efficient process flow, process steps a) and b) can take place in a “one-pot” reaction in one and the same reaction solution. Surprisingly, it has been shown that the proposed process steps can be carried out with high efficiency under the same reaction conditions. By coupling the two substeps in one solution, the procedural effort can be kept low. Significant reaction rates and high space-time yields result. This is particularly astonishing since competitive inhibition or other negative effects in enzymatic performance had to be expected with the partial enzymatic conversions proposed here. Surprisingly, however, these were not observed, or only to a small extent, and a synergistic increase in performance can also result from the coupling of both systems in solution.

In a preferred process design, process steps a) and b) can be carried out simultaneously. It has also been found to be particularly advantageous that both reaction steps are carried out simultaneously. Both steps are carried out simultaneously in that at least partial or total amounts of both enzymes are present in a common reaction solution at the start of the reaction and the intermediate product formed is always in equilibrium with both enzyme systems. The simultaneous process design explicitly does not include that both partial steps are carried out at the same rate or that the same amount of intermediate/final product is always obtained in terms of time.

Within another preferred aspect of the process, the pH in process step a) and/or b) may be greater than or equal to 5.0 and less than or equal to 8.5. Surprisingly, it has been shown that both process step a) and process step b) can be carried out with high conversions within a relatively limited range of pH values. The reaction rates of the substeps of the enzyme cascade presented here thus lie in the same subranges of the pH spectrum and thus allow the preferred simultaneous execution of the subreactions in only one aqueous process solution without a significant reduction of the reaction rates in the individual substeps.

In a preferred embodiment of the process, process step a) and/or b) can be carried out in the presence of fructose-1,6-bisphosphate at a concentration of greater than or equal to 5 μM and less than or equal to 200 μM. To increase the reaction rate and to improve ATP regeneration, the addition of fructose-1,6-bisphosphate in the concentration indicated above has proven to be particularly suitable. The addition accelerates not only one but both partial steps of the reaction, which was not to be expected in this way. In this respect, the addition can in particular improve the performance of the reaction in only one reaction solution and increase the ATP regeneration performance.

Within a preferred embodiment of the process, the inorganic pyrophosphate can be formed in process step b) by an enzyme-catalyzed reaction in the aqueous solution. To control the reaction within the cascade, it has been found to be particularly advantageous that the inorganic pyrophosphate is formed in situ and not added separately from the outside. This has the advantage that the local PPᵢ quantities can be kept small, which in turn improves the catalytic performance of the enzymes by reducing inhibitory substances.

According to a preferred embodiment of the process, the glucose-1-phosphate formed in process step b) can be removed from the aqueous solution by a further enzymatic reaction. In order to increase ATP synthesis, it has been found to be particularly advantageous that parts of the products are removed from the equilibrium by a further enzymatic reaction. This removal can be achieved, for example, by a further enzyme-catalyzed step with an enzyme selected from the group consisting of phosphoglucomutase, glucose 1-phosphatase, nucleoside triphosphate monosaccharide 1-phosphate nucleotidylyltransferases, uridine triphosphate monosaccharide 1-phosphate uridylyltransferase, disaccharide phosphorylases such as sucrose phosphorylase or trehalose phosphorylase, or mixtures of at least two of these enzymes. These enzymes have proved to be particularly suitable for this task and interact only to a small extent with the other cascade steps, so that a one-pot reaction can also be carried out here without reducing the basic yield.

Furthermore, according to the invention, the use of the process according to the invention for the in-situ provision of adenosine triphosphate in multistage, adenosine triphosphate-consuming enzyme cascades in the production of compounds selected from the group consisting of sugar phosphates, nucleotide sugars, glycans, glycoproteins, glycolipids, glycosaminoglycans, phospho-adenosine phosphosulfate, nucleotide-activated compounds or mixtures of at least two compounds from this group. The present provision of ATP by the regeneration of ATP degradation products may be particularly suitable for the production of the above substances in larger enzyme cascades. The two-step conversion is robust and tolerant to the presence of further enzymes and compounds and, in this respect, a long-lasting, controlled ADP work-up can be carried out without the need to add new enzyme or ATP to the system. Moreover, the proposed cascade is applicable under different environmental conditions, so that ATP regeneration can be used as a flexible building block in the context of substance synthesis. Moreover, the required regeneration rate can be precisely adjusted via the choice of the individual enzyme concentrations. In this respect, an additional increase in synthesis performance can be achieved via a simple addition of the cascade according to the invention to the synthesis cascades. Overall, the regeneration cascade can contribute to an extension of the service life of the synthesis cascades and to an optimization of the space-time yields.

Within a preferred embodiment of the use, the adenosine triphosphate-consuming enzyme reaction may comprise the conversion of N-acetylglucosamine (GlcNAc) and adenosine triphosphate (ATP) to N-acetylglucosamine 1-phosphate (GlcNAc-1-P) and adenosine diphosphate (ADP) by means of an N-acetylhexosamine 1-kinase (BlNahK). The proposed ATP regeneration can enable particularly efficient syntheses, especially with partial or total cascades in the context of enzymatic hyaluronic acid production. For example, the above-described conversion of N-acetylglucosamine according to the following equation can precede or parallel the equilibrium of the proposed two-step cascade:

GlcNAc+ATP↔GlcNAc-1-P+ADP.

The reaction is enzyme-catalyzed by means of BlNahK with consumption of ATP to ADP. The consumed ATP can be returned to the system via conversion or by using PPᵢ. This regeneration can extend the run times of the synthesis without addition of further ATP. The overall reaction in the context of this use, including the described ATP regeneration, can be represented as follows:

GlcNAc+PPᵢ+Suc↔GlcNAc-1-P+Glc-1-P+Fru.

In a preferred embodiment of use, the N-acetylglucosamine-1-phosphate can be converted in a further enzymatic reaction using uridine triphosphate by means of a uridine diphosphate-N-acetylglucosamine diphosphorylase to uridine diphosphate-N-acetylglucosamine and inorganic pyrophosphate. In order to shift the equilibrium of the individual enzymatic steps, it has been found to be particularly advantageous that, for example, for hyaluronic acid synthesis, the formed N-acetylglucosamine-1-phosphate is removed from the system in the course of a further reaction. In addition to shifting the equilibrium, the type of removal described here also achieves that PPᵢ is specifically generated in the process. This PPᵢ can be used as reactant in the subsequent steps, so that at best the addition of further PPᵢ can be omitted. This sub-step can be represented by the following equation:

GlcNAc-1-P+UTP↔UDP-GlcNAc+PPᵢ.

In the context of an overall consideration of the 4-step system including the two steps of ATP regeneration, the following overall reaction equation can be obtained:

GlcNAc+Suc+UTP↔UDP-GlcNAc+Fru+Glc-1-P.

In a further preferred aspect of the use, the glucose-1-phosphate formed in process step b) can be converted to uridine 5′-diphosphoGlucose and inorganic pyrophosphate by further enzymatic conversion using uridine triphosphate with a uridine triphosphate monosaccharide-1-phosphate uridylyltransferase. In general, regardless of the upstream cascade steps, it may be useful for the Glc-1-P formed in the final step of regeneration to be removed from equilibrium. It has proven to be particularly efficient to also perform this step enzymatically on the basis of a uridylyltransferase. This step not only removes product from the equilibrium and shifts the equilibrium towards products, it also simultaneously provides PPᵢ for further reaction. This can reduce overall production costs and contribute to on-demand PPᵢ supply. A possible reaction equation for this single step results in:

Glc-1-P+UTP↔UDP-Glc+PPᵢ.

Within an overall consideration with the upstream cascade steps for the production of hyaluronic acid or derivatives, the following equation can result:

GlcNAc+Suc+2UTP↔UDP-GlcNAc+Fru+UDP-Glc+PPᵢ.

In a preferred aspect of the use, the glucose-1-phosphate formed in process step b) can be converted to glucose-6-phosphate by further enzymatic reaction with a phosphoglucomutase. Another way to convert the Glc-1-P in the reaction solution by an enzymatic step is via the use of a phosphoglucomutase. This enzyme can be used in the same reaction solution of the previous steps and in this respect it results in a flexible and efficient ATP regeneration system. This conversion can be represented as:

Glc-1-P↔Glc-6-P.

In the context of the overall consideration of the use, the following overall equation results:

GlcNAc+Suc+UTP↔UDP-GlcNAc+Fru+Glc-6-P.

Further advantages and advantageous embodiments of the objects according to the invention are illustrated by the examples and drawings and explained in the following description. It should be noted that the drawings are descriptive only and are not intended to limit the invention.

The figures show:

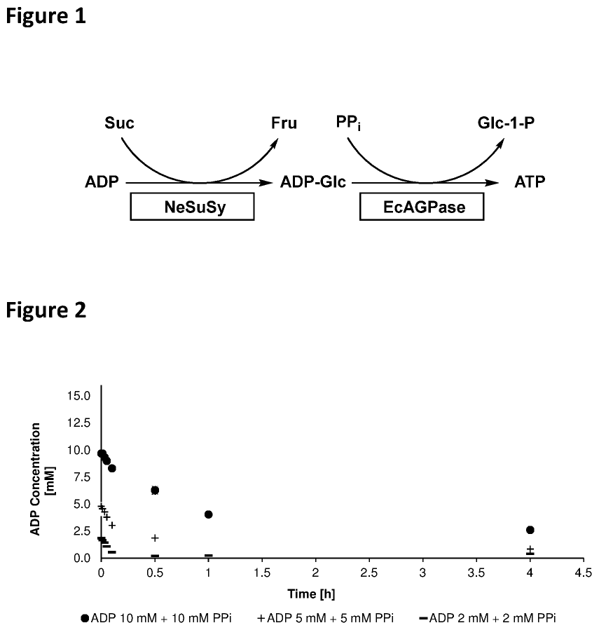

FIG. 1 a two-step enzyme cascade according to the invention;

FIG. 2-4 the analytical results of the two-step enzyme cascade;

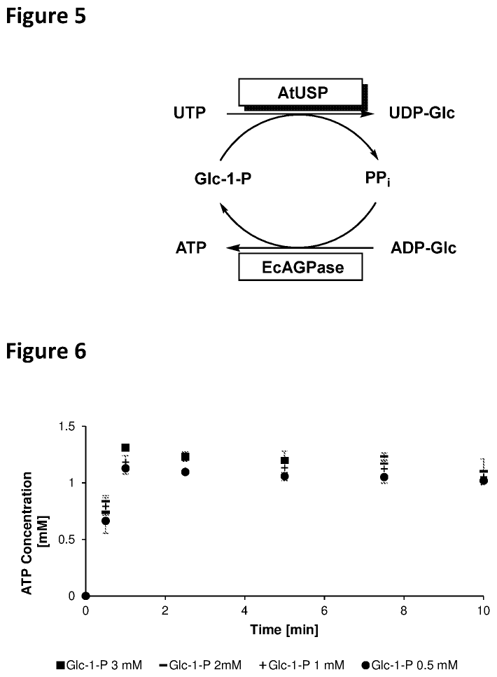

FIG. 5 a two-step enzyme cascade according to the invention with in-situ generation of PPᵢ;

FIG. 6-9 the analytical results of the two-step enzyme cascade;

FIG. 10 a 3-step cascade based on the individual steps according to the invention;

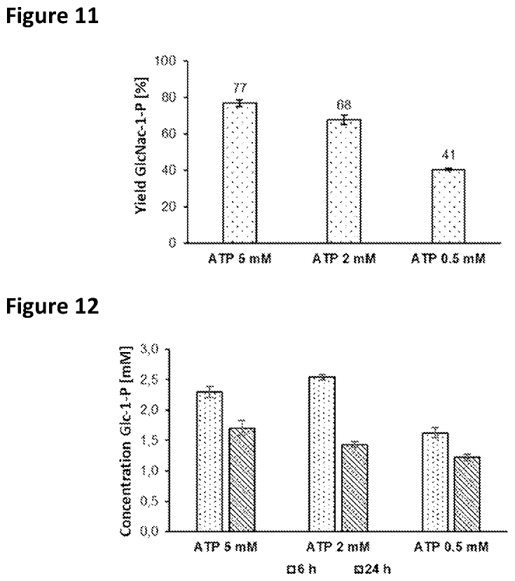

FIG. 11-13 the analytical results of the three-step enzyme cascade;

FIG. 14 a 4-step cascade based on the individual steps according to the invention;

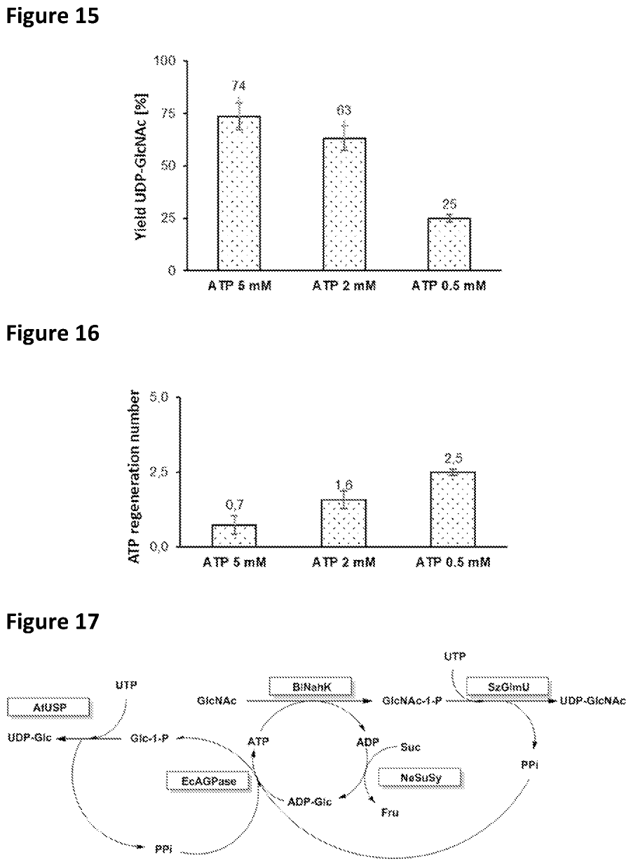

FIGS. 15-16 the analytical results of the 4-step enzyme cascade;

FIG. 17 a 5-step cascade based on the individual steps according to the invention;

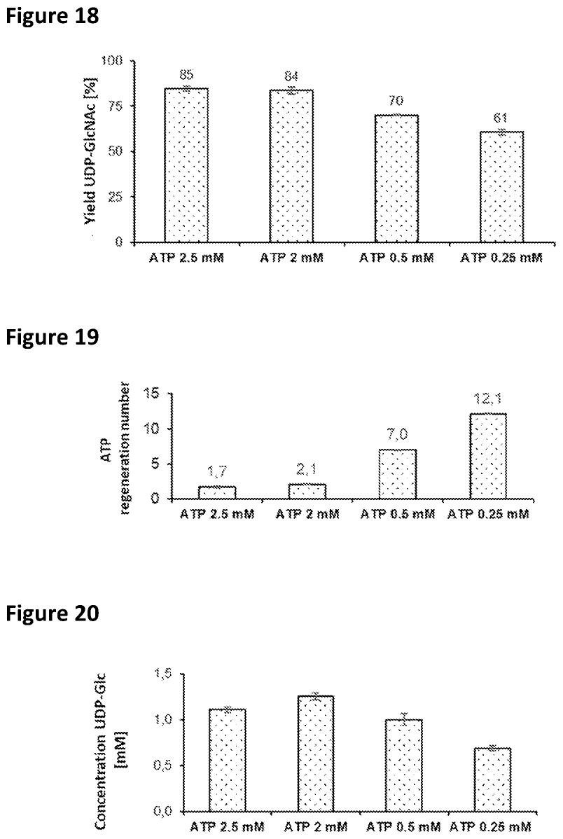

FIG. 18-21 the analytical results of the 5-step enzyme cascade;

FIG. 22 a 5-step cascade based on the individual steps according to the invention.

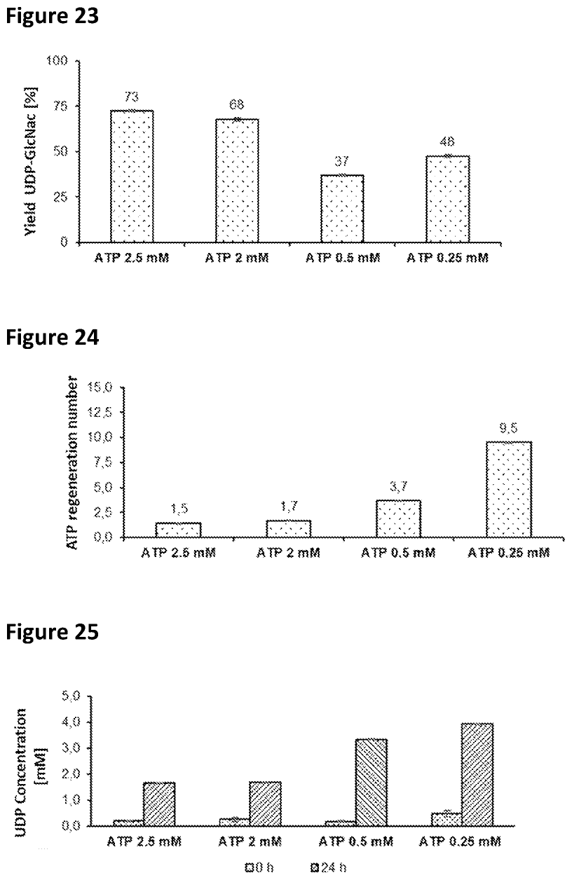

FIG. 23-25 the analytical results of the 5-step enzyme cascade;

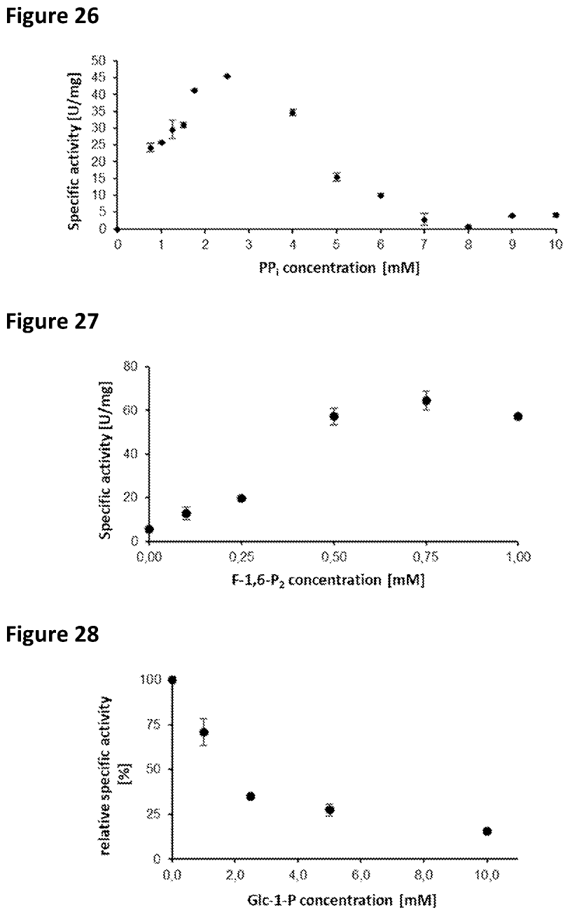

FIGS. 26-28 the analytical results for characterization of EcAGPase.

### EXAMPLES

### I. The Enzymes Used

In the following examples, the following enzymes were used:

| | EC- | | MWCO | Vector | Production | |
|---|---|---|---|---|---|---|
| Enzyme | Number | Origin | [kDa] | pET | master | Tag |
| EcAGPase | 2.7.7.27. | E. coli | 49 | 22b | E. coli | His₆ |
| CgAGPase | 2.7.7.27. | Corynebacterium | 44 | 22b | BL21 (DE3) | |
| | | glutamicum | | | | |
| NeSuSy | 2.4.1.13. | Nitrosomonas | 91 | 22b | | |
| | | europea | | | | |
| AtUSP | 2.7.7.64. | Arabidopsis | 68 | 16b | | |
| | | thaliana | | | | |
| SzGlmU | 2.7.7.23. | Streptococcus equi | 49 | 22b | | |
| | | zooepidemicus | | | | |
| BlNahK | 2.7.1.162. | Bifido- | 40 | 22b | | |
| | | bacterium longum | | | | |
| PmPpA | 3.6.1.1. | Pasteurella | 19 | 22b | | |
| | | multocida | | | | |

Cells were transformed with the appropriate vector via heat shock and proteins were expressed in “terrific broth” (TB) medium overnight using isopropyl-β-D-thiogalactopyranoside (IPTG) induction. Cell disruption was performed by sonication and the expressed enzymes were purified by Ni²⁺-immobilized metal ion affinity chromatography (IMAC) on HisTrap™ HP columns (GE Healthcare, Chicago, USA) on an AKTApurifier™ (GE Healthcare, Chicago, USA) system. Subsequently, the eluate was dialyzed using dialysis tubing (C. Roth, Karlsruhe, Germany) overnight in the respective storage buffer of the enzyme. EcAGPase and CgAGPase were stored in 100 mM HEPES pH 8; NeSuSy in 100 mM Tris-HCl pH 7 and the remaining enzymes in 100 mM HEPES pH 7.5. Protein concentrations of the eluates were performed after dialysis by Bradford assay using RotiQuant solution (C. Roth, Karlsruhe, Germany).

### II. The Two-Step Enzyme Cascade According to the Invention

The reaction scheme of ATP synthesis reaction from sucrose (Suc) and inorganic pyrophosphate (PPᵢ) by coupling EcAGPase and CgAGPase results in substeps a) and b) and in the overall summary to:

ADP+Suc ↔ADP-Glc+Fru a)

ADP-Glc+PPᵢ↔Glc-1-P+ATP b)

ADP+Suc+PPᵢ↔ATP+Fru+Glc-1-P Σ)

The interaction of the individual enzymatic cascade elements is shown in FIG. 1.

EcAGPase (2.9 mg/mL) and NeSuSy (0.1 μg/mL) are combined in a one-pot synthesis to synthesize ATP from sucrose and PPᵢ. For this purpose, ADP and PPᵢ are present in the experimental series in a concentration ratio of 1:1 at different concentrations (2 mM to 15 mM), respectively. In addition, the synthesis batch contains a MgCl₂ concentration corresponding to the sum of the concentration of ADP and PPᵢ in the respective experiment (4 mM to 30 mM). In addition, 1 mM fructose bisphosphate (Fru-1,6-P₂) is added to the reaction. The batch is buffered with 100 mM MOPS-NaOH buffer at pH 8 and ATP synthesis is performed at 37° C. The synthesis is stopped at the respective measurement points with 28 mM SDS (final concentration 7 mM) and the analysis of the nucleotides (ADP-Glc, AMP, ADP, ATP) is performed by multiplex capillary electrophoresis (internal standards (final concentrations): 1 mM para-aminobenzoic acid and 1 mM 4-aminophthalic acid). The analytes are detected at 254 nm.

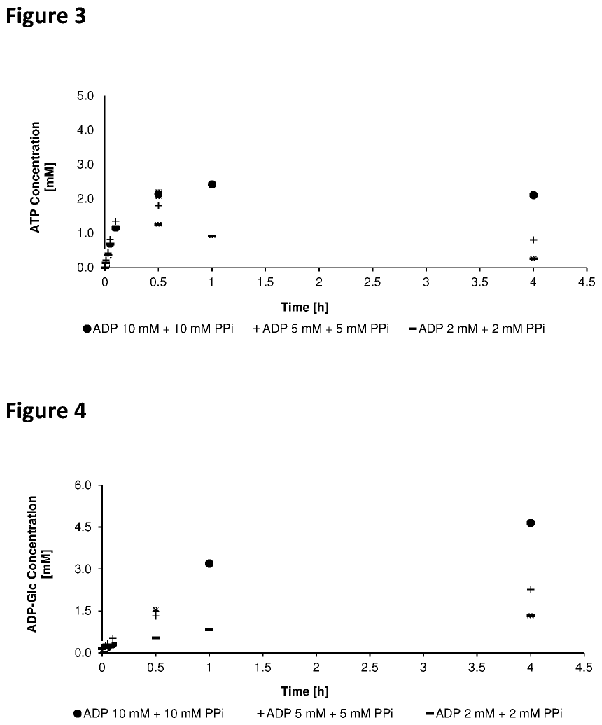

The analytical results of this conversion are shown in FIGS. 2-4. FIG. 2 shows the course of the ADP concentration, FIG. 3 the course of the ATP concentration and FIG. 4 the course of the ADP-Glc concentration over time.

The reaction shown in FIGS. 2-4 contained 2930 μg/mL EcAGPase, 0.1 μg/mL NeSuSy, a gradient of ADP and PPᵢ (1 mM to 15 mM), while the concentrations of sucrose (200 mM) and the activator F-1,6-P₂ (1 mM) were kept constant. The concentrations of MgCl₂ were equal to the sum of the concentrations of ADP and PPᵢ. The reaction was carried out at 37° C. in 100 mM MOPS buffer, pH 7 for 24 h.

The combination of NeSuSy with EcAGPase in an enzyme cascade for ATP synthesis from sucrose and PPᵢ shows that after only a few minutes, the concentration of ADP in the reaction decreases and ATP is formed. However, the synthesis of ATP reaches an ATP synthesis limit between 1.96 mM and 2.14 mM after 30 min of reaction time at ADP and PPᵢ starting concentrations of 5 mM and 10 mM, respectively, corresponding to ATP yields of 39% (5 mM ADP/PPᵢ) and 21% (10 mM ADP/PPᵢ), respectively. The experiments with EcAGPase show that the synthesis of ATP from PPᵢ and ADP-glucose is subject to the reaction equilibrium of EcAGPase (FIG. 1). From the course of ATP synthesis it can be seen (FIG. 3) that ADP-Glc can be converted very rapidly into ATP by EcAGPase in the presence of PPᵢ. However, the example of the experiment with 2 mM ADP/PPᵢ shows that ATP is converted again over a longer period (4 h) when the ADP and PPᵢ concentrations decrease. During the same period, the ADP-Glc concentration increases (FIG. 4). In ATP synthesis, Glc-1-P is also produced along with ATP and leads to the increase of ADP-Glc concentration in the reaction with increasing Glc-1-P concentration. This means that the enzyme EcAGPase sets the reaction equilibrium with increasing Glc-1-P and synthesizes less ATP.

### II. In-Situ Generation of PPᵢ

This reaction sequence involves the in-situ generation of PPᵢ through the use of a complex cascade involving the use of an AtUSP.

This ATP synthesis reaction can be represented as follows:

Glc-1-P+UTP ↔UDP-Glc+PPᵢ

ADP-Glc+PPᵢ↔Glc-1-P+ATP

ADP-Glc+UTP ↔UDP-Glc+ATP

The interaction of the individual enzymatic cascade elements is shown in FIG. 5.

FIGS. 6-9 show the analytical results of the above implementation.

AtUSP and EcAGPase are combined in a one-pot synthesis to use PPᵢ formed in the UDP-Glc synthesis for the synthesis of ATP in a subsequent reaction. The resulting Glc-1-P is again used by AtUSP for the synthesis of UDP-Glc. This attenuates the back reaction of EcAGPase towards ADP-Glc synthesis. This allows a more accurate description of the effect of Glc-1-P on ATP synthesis.

The experimental setup is as follows: 2.9 mg/mL EcAGPase and 0.5 mg/mL AtUSP are combined in a reaction with 3 mM ADP-Glc, 3 mM UTP, and 1 mM F-1,6-P₂. UDP-Glc is synthesized with starting concentrations ranging from 0.5 mM to 10 mM Glc-1-P over a 10 min period at 37° C. in 100 mM HEPES buffer (pH 8). In addition, the reaction contains MgCl₂ whose concentration was adjusted according to the sum of the concentrations of Glc-1-P and UTP. The synthesis is stopped at the respective measurement points with 28 mM SDS (final concentration 7 mM) and the analysis of the nucleotides (ADP-Glc, AMP, ADP, ATP, UTP, UDP, UMP, UDP-Glc) is performed by multiplex capillary electrophoresis (internal standards (final concentrations): 1 mM para-aminobenzoic acid and 1 mM 4-aminophthalic acid) and the analytes are detected at 254 nm.

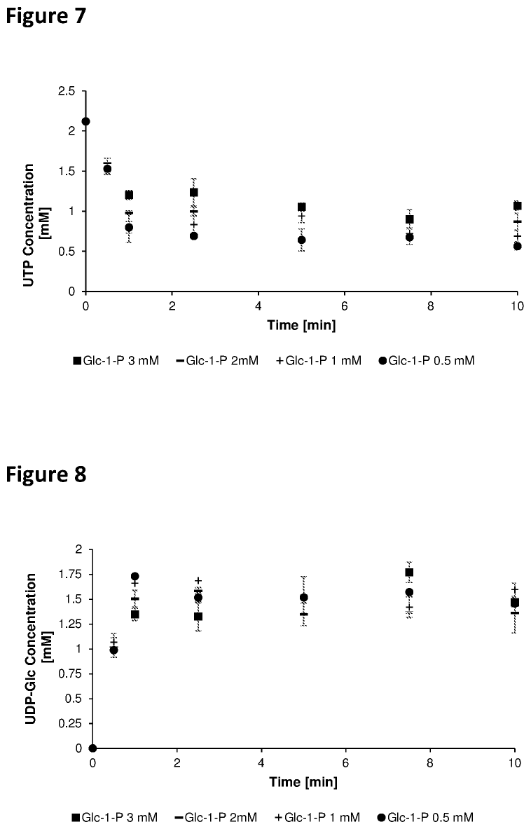

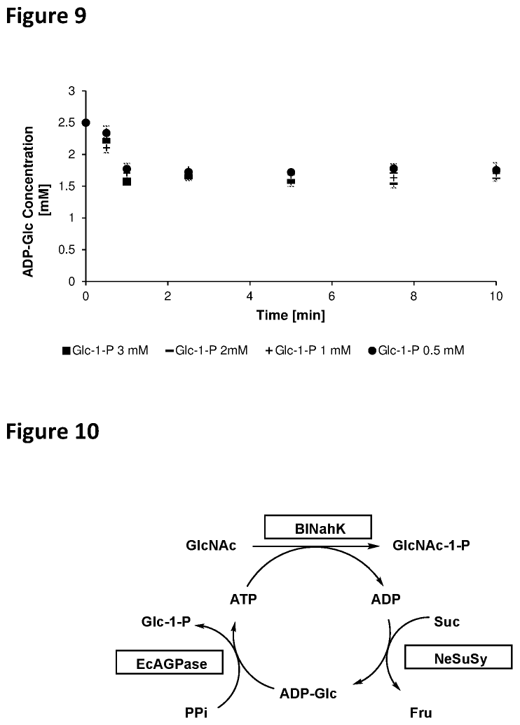

The combination of AtUSP and EcAGPase shows that ATP is formed from the PPᵢ of the UDP-Glc synthesis reaction and ADP-Glc (FIG. 6). In addition, it is shown that all reactions reach a synthesis limit of 1.2 mM ATP (approx. 40% yield relative to UTP) after only one minute and are hardly influenced by the initial Glc-1-P concentration. It is remarkable that with 0.5 mM Glc-1-P an ATP concentration of 1.1 mM is already reached after one minute. This is also reflected by the very rapid conversion of UTP (FIG. 7) and the very rapid synthesis of UDP-Glc (FIG. 8). On the one hand, this means that PPᵢ is used by the AGPase for the synthesis of ATP from ADP-Glc (FIG. 9) after the initial Glc-1-P concentration has been converted. On the other hand, this means that the Glc-1-P formed is in turn used by the enzyme AtUSP for UDP-Glc synthesis. This creates a closed loop in which ATP is synthesized from ADP-Glc, UTP, and in situ generated PPᵢ. However, even here, ATP synthesis is subject to the reaction equilibrium of EcAGPase when the experimental time is prolonged. The concentration of ATP decreases again in the course of the experiment (FIG. 6). ATP should therefore be removed from the synthesis reaction as quickly as possible by combining it with ATP-consuming enzymes in order to maintain the regeneration cycle.

Through this experiment, it is demonstrated that EcAGPase is able to synthesize ATP from in situ nascent PPᵢ, in a coupled enzyme reaction. ATP can in turn be converted as a substrate by ATP-utilizing enzymes.

### III. Three-Step Enzyme Cascade for the Synthesis of GlcNAc-1-P

This reaction sequence involves the BlNahK/NeSuSy/EcAGPase 3-enzyme cascade for the synthesis of GlcNAc-1-P using the new ATP regeneration system.

This ATP synthesis reaction can be represented as follows:

GlcNAc+ATP ↔GlcNAc-1-P+ADP

ADP+Suc ↔ADP-Glc+Fru

ADP-Glc+PPᵢ↔Glc-1-P+ATP

GlcNAc+PPᵢ+Suc ↔GlcNAc-1-P+Fru+Glc-1-P

The interaction of the individual enzymatic cascade elements is shown in FIG. 10.

FIGS. 11-13 show the analytical results of the above implementation.

Reactions were based on 1.6 mg/mL BlNahK, 1.3 mg/mL EcAGPase, 25 μg/mL NeSuSy, 5 mM GlcNAc, 200 mM sucrose, 0.5 mM to 5 mM ATP, 5 mM PPᵢ, 10 mM MgCl₂, and 0.5 mM Fru-1,6-P₂. The experiment was performed in 100 mM MOPS buffer, pH 7 at 37° C. for 24 h. GlcNAc-1-P and Glc-1-P were reacted separately to give UDP-GlcNAc and UDP-Glc, respectively, and measured by MP-CE. Here, 250 μg/mL AtUSP; 2.5 mg/mL SzGlmU and 2.6 mg/mL PmPpA with 10 mM UTP and 10 mM MgCl₂ in 100 mM MOPS buffer, pH 7 at RT for 2 h were added to the reaction after removing the synthesis enzymes. ATP regeneration was calculated using the following formula:

$$Reg {.}_{[ ATP ]} = \frac{{c}_{[ UDP - G 1 cNAc ]}}{{c}_{[ ATP ]}} .$$

The new ATP regeneration system NeSuSy/EcAGPase is combined with an ATP-consuming enzyme, such as a sugar-1-phosphate kinase (using BlNaHK as an example), for the synthesis of GlcNAc-1-P. ATP is consumed by the kinase BlNahK to form GlcNAc-1-P. The resulting ADP is converted by NeSuSy with sucrose to ADP-Glc and fructose. ADP-Glc is subsequently converted to Glc-1-P and ATP by EcAGPase with the addition of PPᵢ. In this way, ATP is made available again (regenerated) from ADP for the sugar kinase reaction.

The experimental setup is as follows: The reaction contains 1.6 mg/mL BlNahK, 1.3 mg/mL EcAGPase and 25 μg/mL NeSuSy, 5 mM GlcNAc, 200 mM sucrose, 0.5 mM to 5 mM ATP, 5 mM PPᵢ and 10 mM MgCl₂ and 0.5 mM Fru-1,6-P₂. Enzyme reactions are performed in 100 mM MOPS buffer, pH 7 at 37° C. for 24 h in a 96-microtiter plate in a volume of 200 μL. After each measurement time point, 150 μL is removed from each of the reaction mixtures and the enzymes are separated from the reaction by ultrafiltration (30 K filter, cut-off 30 kDa, AcroPrep™ Advance filters; Pall) for 15 min. Then, to 100 μL of sample, 50 μL of a solution of AtUSP, SzGlmU, PmPpA, 10 mM UTP and 10 mM MgCl₂ are added to synthesize the nucleotide sugars UDP-GlcNAc and UDP-Glc. These are then analyzed by capillary electrophoresis. The reaction of the enzymes from the follow-up reaction for nucleotide sugar synthesis are stopped with 28 mM SDS (final concentration 7 mM) and the analysis of the nucleotides (ADP-Glc, ADP, ATP, UTP, UDP, UDP-Glc and UDP-GlcNAc) is performed by multiplex capillary electrophoresis (internal standards (final concentrations): 1 mM para-aminobenzoic acid and 1 mM 4-aminophthalic acid) and the analytes detected at 254 nm. The UDP-GlcNAc concentration is proportional to the GlcNAc-1-P formed. The UDP-Glc concentration is proportional to the ATP formed.

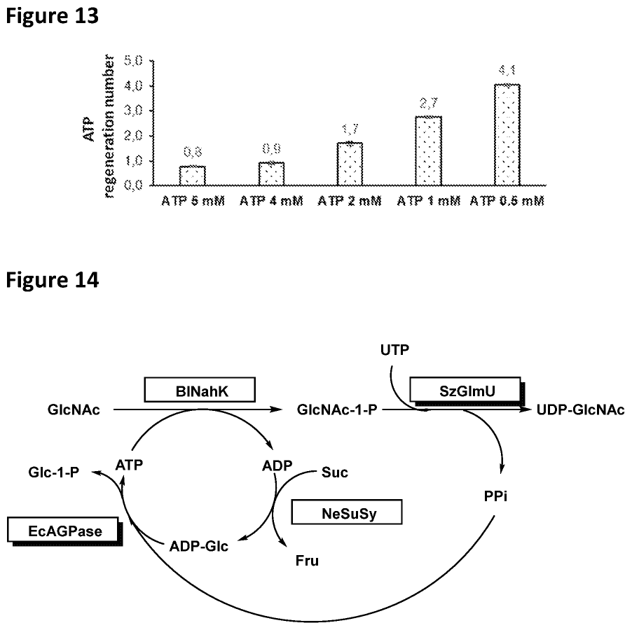

Reduction of the ATP concentration (2 mM and 0.5 mM) below the substrate concentration (5 mM GlcNAc) shows that GlcNAc is phosphorylated with 41%-68% yield (FIG. 11). The control reaction with 5 mM ATP gives a yield of 77%. ATP is regenerated 1.7 and 4.1 times in the reactions with an initial concentration of 2 mM and 0.5 mM, respectively, in the NeSuSy/AGPase enzyme cascade (FIG. 13). Reduction of ATP concentration by more than 50% (from 5 mM to 2 mM) leads to similar product yields. The detected yield of Glc-1-P after 24 h indicates ATP regeneration over 24 h, which also occurred in reactions with ATP excess. Furthermore, these results support the assumption that during the production process of GlcNAc-1-P, ATP is recycled. However, it also shows that Glc-1-P undergoes saturation during ATP recycling and is degraded during the synthesis process (FIG. 12). Four out of a maximum of ten ATP regeneration cycles (at 0.5 mM ATP) are achieved (coupling efficiency 40%). A further increase is possible by optimizing the enzyme ratios.

In combination with a sugar kinase (ATP-consuming enzyme), the new ATP regeneration system NeSuSy/AGPase is capable of regenerating ATP from ADP with sucrose and PPᵢ.

### IV. 4-Enzyme Cascade for UDP-GlcNAc Synthesis with ATP Regeneration System According to the Invention

This ATP synthesis reaction using a 4-enzyme cascade BINahK/SzGlmU/NeSuSy/EcAGPase to synthesize UDP-GlcNAc with new ATP regeneration system can be shown as follows:

GlcNAc+ATP ↔GlcNAc-1-P+ADP

GlcNAc-1-P+UTP ↔UDP-GlcNAc+PPᵢ

ADP+Suc ↔ADP-Glc+Fru

ADP-Glc+PPᵢ↔Glc-1-P+ATP

GlcNAc+Suc+UTP ↔UDP-GlcNAc-1-P+Fru+Glc-1-P

The interaction of the individual enzymatic cascade elements is shown in FIG. 14.

FIGS. 15 and 16 show the analytical results of the above implementation.

The ATP regeneration system NeSuSy/EcAGPase is combined with the enzyme cascade BlNahK/SzGlmU for the synthesis of UDP-GlcNAc. GlcNAc is converted to GlcNAc-1-P and ADP with BlNahK consuming ATP. GlcNAc-1-P is then converted with SzGlmU to UDP-GlcNAc with release of PPᵢ. NeSuSy converts ADP and sucrose to ADP-Glc and fructose. EcAGPase uses the released PPᵢ and ADP-Glc to form Glc-1-P and ATP, which is thus regenerated.

The experimental setup is as follows: The synthesis reaction contains 57.5 μg/mL EcAGPase, 58 μg/mL NeSuSy, 84 μg/mL BlNahK, 94 μg/mL SzGlmU, 5 mM UTP, 200 mM sucrose, 0.5 mM Fru-1,6-P₂, 10 mM MgCl₂ and the ATP concentration is 0.5 mM to 5 mM. Reactions are performed on a 200 μL scale in a 96-microtiter plate at 37° C. for 24 h in 100 mM HEPES buffer pH 7. With each measurement time point, 150 μL of a reaction is removed and the enzymes are separated from the reaction by ultrafiltration (30 K filter, cut-off 30 kDa, AcroPrep™ Advance filters; Pall) for 15 min. To determine the resulting Glc-1-P concentration, 50 μL of a solution of AtUSP, PmPpA, 10 mM UTP, and 10 mM MgCl₂ are then added to 100 μL of sample. The resulting nucleotide sugar UDP-Glc is analyzed by capillary electrophoresis. The UDP-Glc concentration is proportional to the ATP formed. The reaction of the enzymes from the follow-up reaction to the nucleotide sugar synthesis are stopped with 28 mM SDS (final concentration 7 mM) and the analysis of the nucleotides (ADP-Glc, ADP, ATP, UTP, UDP, UDP-Glc and UDP-GlcNAc) is performed by multiplex capillary electrophoresis (internal standards (final concentrations): 1 mM para-aminobenzoic acid and 1 mM 4-aminophthalic acid) and analytes are detected at 254 nm.

Reduction of ATP concentration (2 mM and 0.5 mM) below the substrate concentration (5 mM GlcNAc) shows that UDP-GlcNAc is synthesized with 25%-63% yield (FIG. 15). The control reaction with 5 mM ATP gives a yield of 74%. ATP is regenerated 1.6 and 2.5 times in the reactions with an initial concentration of 2 mM and 0.5 mM, respectively, in the NeSuSy/AGPase enzyme cascade (FIG. 16). Reduction of ATP concentration by more than 50% (from 5 mM to 2 mM) leads to similar product yields. Almost three out of a maximum of ten ATP regeneration cycles (at 0.5 mM ATP) are achieved (coupling efficiency 30%). Further increase should be possible by optimizing the enzyme ratios and by removing Glc-1-P from the reaction equilibrium of EcAGPase.

In combination with a sugar kinase (ATP-consuming enzyme) and a pyrophosphorylase (PPᵢ generating enzyme), the new ATP regeneration system NeSuSy/AGPase is capable of regenerating ATP from ADP with sucrose and PPᵢ.

### V. 5 Enzyme Cascade for UDP-GlcNAc Synthesis

This ATP synthesis reaction using a 5-enzyme cascade BINahK/SzGlmU/NeSuSy/EcAGPase/AtUSP to synthesize UDP-GlcNAc with the ATP regeneration system of the invention can be described as follows:

GlcNAc+ATP ↔GlcNAc-1-P+ADP

GlcNAc-1-P+UTP ↔UDP-GlcNAc+PPᵢ

ADP+Suc ↔ADP-Glc+Fru

ADP-Glc+PPᵢ↔Glc-1-P+ATP

Glc-1-P+UTP ↔UDP-Glc+PPᵢ

GlcNAc+Suc+2 UTP ↔UDP-GlcNAc+Fru+UDP-Glc+PPᵢ

The interaction of the individual enzymatic cascade elements is shown in FIG. 17.

FIGS. 18-21 show the analytical results of the above implementation.

The enzyme cascade for UDP-GlcNAc synthesis is completed with the enzyme AtUSP. Glc-1-P is converted to UDP-Glc with AtUSP and thus removed from the reaction of EcAGPase to suppress the back reaction of EcAGPase and provide more ATP for the enzyme cascade BINahK/SzGlmU. This results in a higher product yield for UDP-GlcNAc synthesis.

The experimental setup is as follows: The synthesis is performed in a one-pot procedure with five enzymes. 41.5 μg/mL NeSuSy, 834 μg/mL BlNahK, 960 μg/mL EcAGPase, 1.2 mg/mL SzGlmU and 75.5 μg/mL AtUSP are used. Synthesis was performed using 5 mM GlcNAc, 10 mM UTP, 0.5 mM F-1,6-P₂, 10 mM MgCl₂, and 0.25 mM to 2.5 mM ATP. The reaction was carried out in 200 μL in 100 mM HEPES pH 7 at 37° C. for 24 h in a 96-microtiter plate. The synthesis was stopped with 28 mM SDS (final concentration 7 mM) and the analysis of nucleotides (ADP-Glc, AMP, ADP, ATP, UTP, UDP, UMP, UDP-Glc, and UDP-GlcNAc) was performed by multiplex capillary electrophoresis (internal standards (final concentrations): 1 mM para-aminobenzoic acid and 1 mM 4-aminophthalic acid) and the analytes were detected at 254 nm.

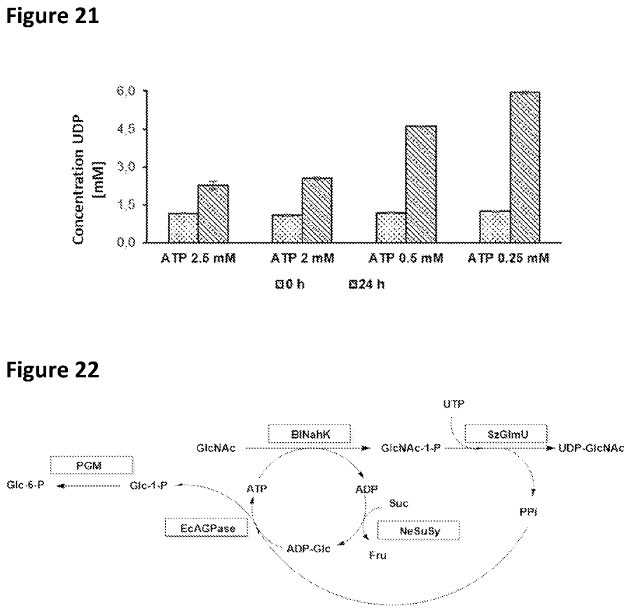

Removal of Glc-1-P from the reaction equilibrium of EcAGPase by reaction with AtUSP significantly increases the yield for UDP-GlcNAc and regeneration of ATP. After 24 h, UDP-GlcNAc yields ranging from 61% (0.25 mM ATP) to 85% (2.5 mM ATP) are achieved (FIG. 18). ATP is regenerated 1.7 and 12.5 times in the reactions with initial concentrations of 2.5 mM and 0.25 mM, respectively, in the NeSuSy/AGPase enzyme cascade (FIG. 19). The UDP-Glc concentration after 24 h shows that this does not correspond to the theoretical maximum concentration of UDP-Glc (12.1 regeneration cycles of 0.25 mM ATP would correspond to 3 mM UDP-Glc, for example). It is expected that with each mole of regenerated ATP, one mole of Glc-1-P is produced by AGPase and subsequently converted to UDP-Glc by AtUSP (FIG. 20). The low amount of UDP-Glc is probably based on the hydrolysis activity of NeSuSy leading to the degradation of UDP-Glc, which is also evidenced by an increasing UDP concentration (FIG. 21). Reducing the ATP concentration by 50% (from 5 mM to 2.5 mM) leads to similar product yields (about 85%). 12 out of a maximum of 20 ATP regeneration cycles (at 0.25 mM ATP) are achieved (coupling efficiency 60%). A further increase should be possible by optimizing the enzyme ratios.

In combination with the UDP-sugar pyrophosphorylase AtUSP (PPᵢ generating enzyme), a sugar kinase (ATP-consuming enzyme) and another pyrophosphorylase (PPᵢ generating enzyme), the new ATP regeneration system NeSuSy/AGPase is capable of regenerating ATP from ADP with sucrose and PPᵢ very efficiently and achieving high product yields. A key function is assigned to the nascent Glc-1-P in the reaction of EcAGPase. Glc-1-P should be removed from the reaction equilibrium. This can be achieved, for example, with the enzyme AtUSP, which converts Glc-1-P with UTP to UDP-Glc and PPᵢ. AtUSP thus additionally forms PPᵢ, which drives ATP synthesis and thus ATP regeneration.

Other enzymes such as sugar P mutases (phosphoglucomutase, formation of Glc-6-P) and sugar phosphate isomerases (Fru-6-P isomerase, formation of Fru-6-P) would also be suitable to remove Glc-1-P from the equilibrium of EcAGPAse.

### VI. Further 5-Enzyme Cascade with ATP Regeneration System According to the Invention

The ATP synthesis reaction can proceed in the context of one of the phosphate-free UDP-GlcNAc synthesis with PGM to remove Glc-1-P according to the following equations:

GlcNAc+ATP ↔GlcNAc-1-P+ADP

ADP+Suc ↔ADP-Glc+Fru

GlcNAc-1-P+UTP ↔UDP-GlcNAc+PPᵢ

ADP-Glc+PPᵢ↔Glc-1-P+ATP

Glc-1-P ↔Glc-6-P

GlcNAc+Suc+UTP ↔UDP-GlcNAc-1-P+Fru+Glc-6-P

The interaction of the individual enzymatic cascade elements is shown in FIG. 22.

FIGS. 23 to 25 show the analytical results of the above implementation.

Using AtUSP to reduce Glc-1-P in the reaction resulted in an additional unit of PPᵢ, which could be used by EcAGPase to regenerate ATP. Therefore, this experiment tests how the system behaves when only one unit of PPᵢ is provided during UDP-GlcNAc synthesis.

The experimental setup is as follows: The synthesis was performed in a one-pot procedure on a 96-well plate with a volume of 200 μL. Each reaction batch contained ATP at different concentrations (0.25 mM-2.5 mM), UTP (7 mM), GlcNAc (5 mM), sucrose (200 mM), F-1,6-P₂ (0.5 mM), and MgCl₂ (10 mM). The reactions were additionally carried out at 37° C. in 100 mM MOPS-buffer pH 7 for 24 h. BlNahK and SzGlmU enzymes were added at concentrations of 0.5 mg/mL and 5 μg/mL, respectively. The enzymes of the ATP regeneration cascade were used at the concentrations of 23.5 μg/mL (NeSuSy) and 175 μg/mL. The enzyme phosphoglucomutase (PGM) from hare muscle (Sigma Aldrich, USA) was added to the cascade at a concentration of 600 μg/mL. The reactions were stopped at the respective measurement points using a stop solution (28 mM SDS, 5 mM PAPA, 1 mM PABA) and analyzed by MP-CE. The nucleotides (ATP, ADP, AMP, UTP, UDP and UMP) and nucleotide sugars (UDP-GlcNAc and ADP-Glc) were detected on MP-CE using UV at 254 nm.

By replacing the AtUSP with PGM, UDP-GlcNAc yields of up to 73% could be achieved from 5 mM GlcNAc and 2.5 mM ATP after 24 h (FIG. 23). Further reduction of the ATP amount resulted in product yields between 60% (2 mM ATP) and at least 37% (0.5 mM ATP). The regeneration of ATP was most evident in the synthesis with 0.25 mM ATP. Here, 48% (2.4 mM) of the GlcNAc used was converted to UDP-GlcNAc, corresponding to a 9.5-fold ATP regeneration (FIG. 24). However, the reduction of the ATP concentration again resulted in an increase of the UDP concentration (FIG. 25).

The use of PGM for the reduction of Glc-1-P shows that coupling of the system according to the invention with further enzymes for Glc-1-P reduction is possible.

### VII. Characterization of AGPase from E. coli

### VII.1 Influence of PPᵢ Concentration

The influence of PPᵢ concentrations on the ATP synthesis activity of the EcAGPase used was investigated.

The experiment included 4.35 μg/mL EcAGPase, 2.5 mM ADP-Glc, 10 mM MgCl₂, 1 mM Fru-1,6-P₂ and PPᵢ, at concentrations ranging from 0.75 mM to 10 mM, and a control reaction without PPᵢ. ATP synthesis was performed in 600 μL of 100 mM HEPES buffer, pH 8 at 37° C. for 12 min. The synthesis was stopped at the respective measurement points with 28 mM SDS (final concentration 7 mM) and the analysis of the nucleotides (ADP-Glc, AMP, ADP, ATP) was performed by multiplex capillary electrophoresis (internal standards (final concentrations): 1 mM para-aminobenzoic acid and 1 mM 4-aminophthalic acid) and the analytes were detected at 254 nm.

EcAGPase already shows a reduction in specific activity at PPᵢ concentrations above 3 mM (FIG. 26). At a PPᵢ concentration of 7 mM, the specific activity of the enzyme is only about 1% (0.6 U/mg) of the activity compared to the non-inhibited reaction (50 U/mg).

EcAGPase is subject to substrate inhibition by PPᵢ in the ATP synthesis direction. Therefore, ATP regeneration by this enzyme may be less efficient in reactions that involve a lot of PPᵢ or release a lot of PPᵢ very rapidly. This disadvantage can be compensated by increasing the EcAGPase concentration.

### VII.2 Influence of Fructose (Fru)-1,6-P₂ Concentration

Fru-1,6-P₂ was used as an activator for EcAGPase. It is Fru-1,6-P₂ a relatively expensive compound and its use in the EcAGPase reaction should be reduced as much as possible. Therefore, the activity of EcAGPase was investigated for different Fru-1,6-P₂ concentrations to determine a minimum concentration of the activator.

The experiment included 3.9 μg/mL EcAGPase, 3 mM ADP-Glc, 3 mM PPᵢ, 10 mM MgCl₂ and Fru-1,6-P₂ at concentrations ranging from 0.1 mM to 1 mM, as well as a control reaction without Fru-1,6-P₂. The synthesis was performed in 600 μL of 100 mM HEPES buffer, pH 8 at 37° C. for 12 min. The synthesis was stopped at the respective measurement points with 28 mM SDS (final concentration 7 mM) and the analysis of the nucleotides (ADP-Glc, AMP, ADP, ATP) was performed by multiplex capillary electrophoresis (internal standards (final concentrations): 1 mM para-aminobenzoic acid and 1 mM 4-aminophthalic acid) and the analytes were detected at 254 nm.

With 0.5 mM Fru-1,6-P₂, a high EcAGPase activity (60 U/mg, 12-fold higher than without activator) is still achieved (FIG. 27). At 0.25 mM, the activity decreases sharply (20 U/mg). EcAGPase still shows an activity of 5 U/mg even in the absence of the activator.

The activator Fru-1,6-P₂ significantly increases the activity of EcAGPase. The concentration of the activator can be significantly reduced. Therefore, it is also possible to perform ATP regeneration with EcAGPase efficiently with very small Fru-1,6-P₂ concentrations and even without activator.

### VII.3 Influence of the Shift of the Reaction Equilibrium

The EcAGPase prefers the ADP-Glc synthesis. Thus, the accumulation of Glc-1-P negatively affects the reaction equilibrium for ATP synthesis. Therefore, the effect of Glc-1-P on the ATP synthesis activity of EcAGPase was investigated.

The experiment included 8.5 μg/mL EcAGPase, 3 mM ADP-Glc, 3 mM PPᵢ, 0.5 mM Fru-1,6-P₂, 10 mM MgCl₂ and Glc-1-P at concentrations ranging from 1 mM to 10 mM, and a control reaction without Glc-1-P. The synthesis was performed in 600 μL of 100 mM HEPES buffer, pH 8 at 37° C. for 12 min. The synthesis was stopped at the respective measurement points with 28 mM SDS (final concentration 7 mM) and the analysis of the nucleotides (ADP-Glc, AMP, ADP, ATP) was performed by multiplex capillary electrophoresis (internal standards (final concentrations): 1 mM para-aminobenzoic acid and 1 mM 4-aminophthalic acid) and the analytes were detected at 254 nm.

EcAGPase shows a strongly reduced ATP synthesis activity at increasing Glc-1-P concentrations (FIG. 28). The activity of the enzyme already drops at 1 mM Glc-1-P to 71% of the activity without the addition of Glc-1-P. With 5 mM Glc-1-P, the residual activity is only 27%. The IC50 value determined for EcAGPase ATP synthesis activity is 1.78 mM Glc-1-P. The Glc-1-P concentration has a strong influence on the efficiency of ATP regeneration by EcAGPase. Therefore, it is recommended for the ATP regeneration system that Glc-1-P is actively removed from the process with increasing cascade duration.

The invention underlying this patent application was developed in a project funded by the BMBF under the grant number 031B0104B.

1. A process for the multistage enzymatic conversion of adenosine diphosphate to adenosine triphosphate, characterized in that the process comprises at least the steps:
(a) enzyme-catalyzed conversion of adenosine diphosphate in the presence of sucrose and a sucrose synthase to adenosine diphosphate-glucose; and
b) Enzyme-catalyzed conversion of the adenosine diphosphate-glucose formed in process step a) in the presence of inorganic pyrophosphate and a pyrophosphorylase to adenosine triphosphate and glucose-1-phosphate;
wherein process steps a) and b) are carried out in aqueous solution and simultaneously or successively, wherein process steps a) and b) are carried out in a common aqueous reaction solution.

2. (canceled)

3. Process according to claim 1, wherein method steps a) and b) are carried out simultaneously.

4. Process according to claim 1, wherein the pH in process step a) and/or b) is greater than or equal to 5.0 and less than or equal to 8.5.

5. Process according to claim 1, wherein process step a) and/or b) is carried out in the presence of fructose-1,6-bisphosphate at a concentration greater than or equal to 5 μM and less than or equal to 200 μM.

6. Process according to claim 1, wherein the inorganic pyrophosphate is formed in process step b) by an enzyme-catalyzed reaction in the aqueous solution.

7. Process according to claim 1, wherein the glucose-1-phosphate formed in process step b) is removed from the aqueous solution by a further enzymatic reaction.

8. Use of the process of claim 1 for in situ provision of adenosine triphosphate in multistage adenosine triphosphate-consuming enzyme cascades in the preparation of compounds selected from the group consisting of sugar phosphates, nucleotide sugars, glycans, glycoproteins, glycolipids, glycosaminoglycans, phospho-adenosine phosphosulfate, nucleotide-activated compounds, or mixtures of at least two compounds from this group.

9. The use according to claim 8, wherein the adenosine triphosphate-consuming enzyme reaction comprises converting N-acetylglucosamine and adenosine triphosphate to N-acetylglucosamine 1-phosphate and adenosine diphosphate by means of an N-acetylhexosamine 1-kinase.

10. The use according to claim 9, wherein the N-acetylglucosamine-1-phosphate is converted in a further enzymatic reaction using uridine triphosphate by means of a uridine diphosphate-N-acetylglucosamine diphosphorylase to uridine diphosphate-N-acetylglucosamine and inorganic pyrophosphate.

11. The use according to claim 10, wherein the glucose 1-phosphate formed in process step b) is converted to uridine 5′-diphosphoGlucose and inorganic pyrophosphate by further enzymatic reaction using uridine triphosphate with a uridine triphosphate monosaccharide 1-phosphate uridylyltransferase.

12. The use according to claim 10, wherein the glucose 1-phosphate formed in process step b) is converted to glucose 6-phosphate by further enzymatic reaction with a phosphoglucomutase.
